(19) United States
(12) Patent Application Publication
(10) Pub. No.: US 2012/0107935 A1
(43) Pub. Date: May 3, 2012

(54) Simplicity in assembling microstructures

(76) Inventor: Stephen liye Chen, El Monte, CA (US)

(21) Appl. No.: 12/925,746

(22) Filed: Oct. 28, 2010

(51) Int. Cl. C12N 5/02; C12M 1/00

(52) U.S. Cl. 435/395; 435/283.1

(57) ABSTRACT

Concept and examples are provided for simplicity in assembling micro-culture structures. A cell culture could be started prior to the formation of a microstructure. Cultured cells are utilized as glue and spacers to measure and assemble the microstructure automatically.

### FIELD OF THE INVENTION

The present invention relates to devices for adhesive cell cultures and more particularly to materials used for assembling a microstructure in a device.

### BACKGROUND OF THE INVENTION

Cell is the basic and the smallest unit of life. It is a building block of living organisms. The size of a cell body is very small, such as 1 micrometer. To form an organism from cells, biological adhesives are required. Special proteins, known as cell adhesion molecules, have been identified as glue of life. They expand from interior to exterior of cell membrane, with their internal ends linking to cytoskeleton and external ends exposing on cell surface. Whenever within a reachable distance, the external ends will contact and form adhesions to other cells or extracellular matrix.

Based on cell adhesion, in vitro cell culture has become one of the most important tools for biomedical researches and industrial manufactures. To conduct cell culture, a well, with a bottom and surrounding walls, is used to hold a culture medium. Cells are placed in the well and used as a reporter to show effects of drug treatments or as a tool to produce proteins. To avoid contamination of cultured cells, wells of cell cultures are usually made cheaply for disposability, which could result in huge differences between in vivo and in vitro. A misleading in vitro cell culture could lead to Invalid data and false conclusion. Therefore, advanced design and precisely fabricated devices are valuable and highly desirable.

Jervis et al, for example, in US patent application Ser. No. 10-582975, proposes a device having a precisely confined space between barriers for constraining cells. To constrain cells properly, a distance between barriers 28 and 22 must be pre-determined and precisely fabricated prior to cell culture. The critical distance must fit the size of a cell, such as 0.05 micrometer, as written in paragraph [0073]. Unfortunately, Jervis et al creates extreme challenges for the maker of the device. It is a common sense to the skilled in manufacture that 0.05 micrometer belongs to Nanotechnology. Special knowledge, sophisticate equipment, and expensive labor are essentially required. Jervis et al fails to answer critical questions. Such as, how to make a device for constraining cells in a variety of sizes? How to fabricate the precise device cheaply disposable after single use? How to ensure the quality of the device within 3% tolerance, which is 0.0015 micrometer? How to measure the tolerance of 0.0015 micrometer during manufacture? These unanswered questions are unavoidable lethal barriers of its feasibility.

Novel concept and strategies in assembling precise microstructures are highly desirable but remains unsolved.

### SUMMARY OF THE INVENTION

It is, therefore, the objects of the invention to propose a novel concept and to teach feasible examples of precise microstructures for cell culture. The advantages of the invention are clearly distinguished as follows:

1. It proposes novel concept of utilizing living cells as builders and building materials.
2. It creates precise microstructure in a device with extreme affordability.
3. It obtains extreme precision without human efforts and errors.

### BRIEF DESCRIPTION OF THE DRAWINGS

FIG. 1a is an illustrative diagram in sectional view showing the formation of a preferred embodiment.

FIG. 1b is an illustrative diagram showing perspective view of the preferred embodiment.

FIG. 1c is an alternative operation of the preferred embodiment

FIG. 2 is an illustrative diagram in sectional view showing the formation of alternative embodiment.

### DETAILED DESCRIPTION OF THE INVENTION

Chemical glues have been used to join two parts into cell culture devices. The two parts must be dried and cleaned for glues to work. Most of the chemical glues are irreversible and toxic to living cells. In contrast to man-made chemical glues, cultured cells produce glues as well. Cell adhesions have become a common sense for decades. These kinds of glues are perfect for living cells because they are natural proteins of the cultured cells. Their adhesion is reversible. They work under liquid environment of culture medium. They are not toxic. And, they are free. These features match exactly to the ideal glue we have been looking for. But, unfortunately, such ideal biological adhesives have been ignored for decades.

It is agreed that simplicity is the true beauty of innovations. This invention proposes a novel concept of using living cells as both builders and building materials to construct precise microstructures without human efforts and errors. Its novelty can be described from two aspects below.

The first novelty of the concept is a delayed time of assembly. Traditionally, a culture device is assembled mechanically by human prior to cell culture. Living cells are simply used as a reporter to show effects of drug treatments. In the invention, the process of cell culture is started prior to the completion of a microstructure. Biological activities of living cells are utilized to produce glues for joining the microstructure.

The second novelty of the concept is the mechanism of pursuing precision. Mechanical tolerance in manufacture industry is unavoidable. In the invention, the bodies of cultured cells are utilized as spacers to measure and control a gap thickness of a microstructure. That is, the thickness of a microstructure is not pre-determined by human. Instead, the thickness settles automatically according to the size of cell bodies under gravity.

### Example One

### Assebmling a Microstructure in a Well

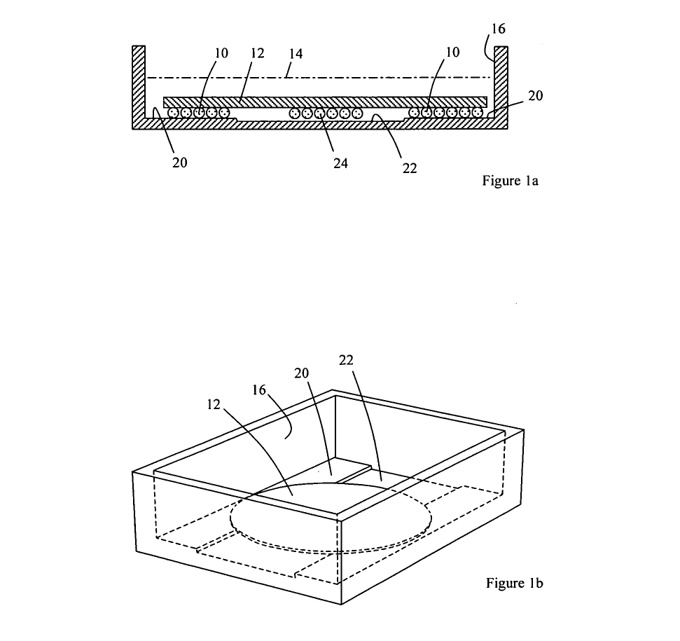

The concept of the invention can be used to construct microstructures in a variety of formats. Microstructure in a well is a preferred example of the invention, as illustrated in FIGS. 1a, 1b, and 1c. For visual illustration, the diagrams are not drawn proportionally to real dimensions because cells are simply too small to be seen. The microstructure can be assembled in steps below:
1. Place cells with culture medium on top of a coverslip 12 (not shown).
2. Let cells to form a first set of adhesions between cells and coverslip 12.
3. Transfer coverslip 12 into well 16 in up side down orientation. Coverslip 12 is a removable foreign piece. It is smaller than the opening and bottom of well 16 for easy placement, as shown in FIGS. 1a and 1b.
4. During cultivation, a second set of adhesions is formed between cells 10 and elevated plates 20 in well 16, as shown in FIG. 1a.

Well 16 has modified bottom features, elevated plates 20 and recessed area 22. Being transferred into well 16 in step 3 above, cells 10 are carried down by coverslip 12 under gravity and held by elevated plates 20. Their direct contact enables formation of a second set of adhesions between cells 10 and elevated plates 20. That is, cells 10 are used as mediators to link coverslip 12 and elevated plates 20 together. Otherwise, coverslip 12 could move freely in well 16. The strength of cell adhesions is relatively fragile and reversible. For securing the anchorage of coverslip 12, elevated plates 20 should be big enough to cover sufficient area of the bottom, such as 50% of the total culture area of well 16. Well 16 is molded with clear polystyrene. Surfaces should be treated by vacuum gas plasma for enhancing attachment of cells.

Recessed area 22 is lower than elevated plates 20 in about 20 micrometers. Cultured cells can be divided into two populations in terms of locations. Cells 10 are first populations located between coverslip 12 and elevated plates 20. The first populations are closed sandwiched and develop two sets of adhesions from both top and bottom sequentially and respectively. Cells 24 are second populations located above recessed area 22. Cells 24 have only one set of adhesions from top. The extra space of recessed area 22 prevents cells 24 from forming unwanted adhesions at bottom. The bottom modification of well 16 creates two microstructures in one culture well under a removable foreign coverslip, which could be beneficial for finding additional information from in vitro optimization.

FIG. 1c is an alternative setup of the preferred embodiment. The operation steps are:
1. Add cells 101 and 241 into well 16 under culture medium 14.
2. Let cells 101 and 241 to form bottom adhesions with elevated plates 20 and recessed area 22 respectively.
3. Place a coverslip 12 into well 16 to contact cells 101 directly so that a second set of adhesions can be formed from top of cells 101 to coverslip 12.

The differences of the alternative setup from the preferred operation are: Cells 241 form adhesions to recessed area 22 instead of coverslip 12. Adhesions of cells 241 are formed from bottom instead of from top.

### Example Two

### Assembling a Micro-Gap Between Two Coverslips

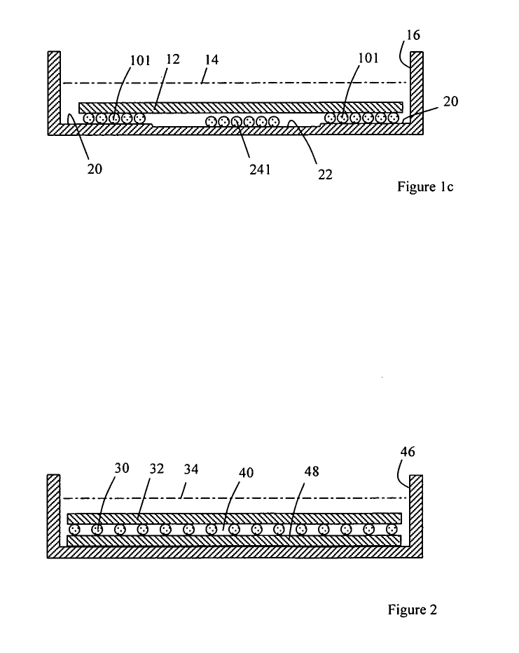

FIG. 2 shows a simplified embodiment of a micro-gap. It can be constructed as follows:
1. Plate cells on top of a coverslip 32 to develop a first set of adhesions between cells 30 and coverslip 32 (not shown).
2. Immerse another coverslip 48 in a well 46.
3. Place coverslip 32 in up side down orientation on top of coverslip 48 to sandwich cells 30, as shown in FIG. 2.
4. During culture, cells 30 develop a second set of adhesions against coverslip 48, which links the two coverslips together.

In the simplified embodiment, gravity drives coverslip 32 towards coverslip 48. Cells 30 are utilized as both adhesives and spacers to construct the micro-gap. As adhesives, cells 30 form two sets of adhesions onto both coverslips sequentially and respectively. As spacers, the bodies of cells 30, after being sandwiched by the two coverslips, prevent coverslip 32 from contacting coverslip 48. The distance between the two coverslips is not pre-determined. A balance between gravity and the size of cell bodies determines the final settlement of the distance. Gravity is a constant. That is, cultured cells can measure and assemble micro-gap precisely and automatically without human efforts and errors.

Gaps 40 are vacant spaces around cells 30. Gaps 40 are filled with culture medium 34 and function as micro-channels of communication. Gaps 40 can generate strong capillary forces to physically change the culture environments of cells 30. Seeding density of cells 30 can be adjusted lower to increase the width of gaps 40.

To summarize the above examples in common:
1. A removable top piece and a bottom piece are used to defining boundaries of a microstructure. There is no direct contact between the removable top piece and a bottom piece. The removable top piece is capable of moving freely in relation to bottom piece.
2. Living cells contact two pieces respectively and mediate their linkage by forming adhesions.
3. Cells are utilized as spacers to determine a distance of microstructures during cell culture.

Although the description above contains specifications, it will apparent to those skilled in the art that a number of other variations and modifications can be made in this invention without departing from its spirit and scope. Coverslip 12, for example, can be made with glass, plastics, semi-permeable membrane, or porous plates. The shapes of coverslip 12 can be circular or rectangular. The thickness of coverslip 12 can be altered as wish. A center hole can be added to coverslip 32. Recessed area 22 can be deeper than 20 micrometers. The size of elevated plates 20 can be much greater than the size of recessed area 22. Recessed area 22 can be very small or omitted. Well 16 can be a single dish of a unit of multi-well plate. Therefore, the description as set out above should not be constructed as limiting the scope of the invention but as merely providing illustration of one of the preferred embodiments of the invention.

1. Adhesives for assembling a microstructure for cell culture, comprising;
a first piece and a second piece to be mediated together for defining boundaries of said microstructure; and
living cells, growing within said microstructure, contacting said first piece and said second piece, forming biological adhesions onto both said first piece and said second piece respectively, linking said first piece and said second piece during said cell culture.

2. The adhesives of claim 1 wherein both said first piece and said second piece are coverslips.

3. Spacers for assembling a micro-gap for cell culture, comprising;
a first piece and a second piece defining boundaries of said micro-gap;
gravity forcing said first piece moving towards said second piece; and
cells, located in said micro-gap, preventing said first piece from contacting said second piece, determining the distance between said first piece and said second piece under said gravity.

4. The spacers of claim 3 wherein said distance of said micro-gap is variable according to growth conditions of said cells.

5. A well with a bottom being modified for micro-cultures of cells, comprising;
an opening of said well for placement of a foreign piece during said micro-culture;
an elevated plate of said bottom, receiving sufficient biological adhesions from said cells under said foreign piece, defining a first structure for said micro-cultures; and
a recessed area, being lower than said elevated plate in a range of micrometers, reserving an extra space under said foreign piece for defining a second structure for said micro-cultures.

6. The well of claim 5 wherein the depth of said recessed area is 20 micrometers lower than said elevated plate.
